(19) United States
(12) Patent Application Publication
(10) Pub. No.: US 2023/0048913 A1
(43) Pub. Date: Feb. 16, 2023

(54) SOLID LIP COSMETIC

(71) Applicant: Shiseido Company, Ltd., Chuo-ku, Tokyo (JP)

(72) Inventors: Misa KANAZAWA, Tokyo (JP); Kyohei KOSAKA, Tokyo (JP)

(73) Assignee: Shiseido Company, Ltd., Chuo-ku, Tokyo (JP)

(21) Appl. No.: 17/790,857

(22) PCT Filed: Dec. 23, 2020
(86) PCT No.: PCT/JP2020/048129

§ 371 Date: Jul. 5, 2022

(30) Foreign Application Priority Data
Jan. 6, 2020 (JP) 2020-000256

(51) Int. Cl. A61K 8/39; A61K 8/25; A61Q 1/04

(52) U.S. Cl. CPC A61K 8/39; A61K 8/25; A61Q 1/04

(57) ABSTRACT

An objective of the invention is to provide a solid lip cosmetic that imparts a highly lustrous finish, while also being resistant to bleeding in the finish after being applied, having excellent stability over time, and further thereto, having a pleasant sense of resilience when applied, being light to spread and providing a high sense of adhesion after application. The solid lip cosmetic according to the present invention contains (A) fatty acid polyglycerides; (B) silicic anhydride having an average particle size of 1 μm or smaller; and (C) a colorant; wherein the component (A) includes (a1) a fatty acid polyglyceride having a viscosity of 5000 mPa·s or higher at 25° C., and (a2) a fatty acid polyglyceride having a viscosity lower than 5000 mPa·s at 25° C.; the component (a1) content is 0.1% to 20% by mass; the mass ratio between components (a1) and (a2) satisfies (a1):(a2)=1:0.5 to 1:5; and the component (B) content is 0.25% to 4% by mass.

### TECHNICAL FIELD

The present invention relates to a solid lip cosmetic. More specifically, the present invention relates to a solid lip cosmetic that has a highly lustrous finish, that has excellent bleeding resistance and stability over time, that has a pleasant sense of resilience when applied, that is light to spread and that provides a high sense of adhesion.

### BACKGROUND ART

Among lip cosmetics, solid lip cosmetics such as lipsticks can be made compact and are easy to carry, and provide the convenience of being able to be directly applied to the lips without using the fingers or tools. Those that can impart particularly high luster can not only achieve a lustrous and attractive appearance, but also strongly reflect light near the center of the lips, with reflected light being difficult to see at both end portions, thereby heightening the sense of depth in appearance and also providing the effect of making the lips appear fuller.

Generally, in order to impart high luster, it is considered to be necessary to increase high-viscosity oils such as hydrogenated polyisobutene and pentaerythritol tetraisostearate. However, if the oil content in a solid lip cosmetic is increased, then the oils tend to spread to the skin on the outer side of the lips after the lipstick has been applied, and there are cases in which bleeding (deterioration of the finish) occurs. Additionally, by increasing the oil content, the hardness of the cosmetic becomes lower, and deformation and breakage tend to occur. For this reason, high-luster lip cosmetics are generally often provided in paste or liquid form, and it is technically difficult to make a solid lip cosmetic that is stable while also being lustrous.

As an attempt to improve the bleeding and stability over time of solid lip cosmetics, for example, Patent Document 1 proposes to combine 10% to 30% by weight of spherical silicic anhydride having an average particle size of 2 to 15 μm, 1% to 3% by weight of aerosol-form silicic anhydride having an average particle size of 5 to 50 nm and a polyglycerin fatty acid ester that is in paste form at 25° C. However, if a large amount of powder is blended, then the cosmetic becomes heavy to spread at the time of application, and also, the luster is suppressed, resulting in a matte finish.

Additionally, there are cases in which bleeding is prevented by thickening the oils by increasing the amount of waxes and the like. However, if the waxes are increased, then the gel can become too hard, making it difficult to apply the cosmetic to the lips, and there are problems such as the dispersion of colorants becoming inadequate, resulting in poor coloration.

### RELATED ART

### Patent Documents

Patent Document 1: JP 2019-108280 A

### SUMMARY OF THE INVENTION

### Problem to be Solved by the Invention

The present invention was made in consideration of the aforementioned circumstances, and an objective of the present invention is to provide a solid lip cosmetic that imparts a high luster, while also being resistant to bleeding in the finish after being applied, having excellent stability over time, and further thereto, having a pleasant sense of resilience when applied, being light to spread and providing an excellent sense of adhesion after application.

### Means for Solving the Problem

The present inventors performed diligent research towards solving the above-mentioned problem, as a result of which they discovered that the above-mentioned problem can be solved by blending two types of fatty acid polyglycerides having prescribed viscosities at a prescribed mass ratio, and also blending a prescribed amount of a fine silicic anhydride having a prescribed average particle size, thereby completing the present invention.

That is, the present invention is basically a solid lip cosmetic containing:

(A) fatty acid polyglycerides;
(B) silicic anhydride having an average particle size of 1 μm or smaller; and
(C) a colorant;

wherein

the component (A) includes

(a1) a fatty acid polyglyceride having a viscosity of 5000 mPa·s or higher at 25° C., and
(a2) a fatty acid polyglyceride having a viscosity lower than 5000 mPa·s at 25° C.;

the component (a1) content is 0.1% to 20% by mass;

the mass ratio between components (a1) and (a2) satisfies (a1):(a2)=1:0.5 to 1:5; and

the component (B) content is 0.25% to 4% by mass.

### Effects of the Invention

By having the above-mentioned features, the solid lip cosmetic according to the present invention can impart high luster to the lips, while also being resistant to bleeding in the finish after being applied, and having adequate stability over time such that the cosmetic does not crumble or soften during storage or use. In addition thereto, the solid lip cosmetic according to the present invention has an appropriate sense of resilience when brought into contact with the lips, is light to spread when applied and provides a high sense of adhesion as if the lips are protected after application.

### MODES FOR CARRYING OUT THE INVENTION

The solid lip cosmetic of the present invention contains (A) fatty acid polyglycerides, (B) silicic anhydride and (C) a colorant, under prescribed conditions. Hereinafter, the components constituting the solid lip cosmetic of the present invention will be explained in detail.

### <(A) Fatty Acid Polyglycerides>

The (A) fatty acid polyglycerides are compounds in which one or more molecules of a fatty acid and two or more molecules of glycerin are bound together. They may be mono fatty acid polyglycerides having a single fatty acid-derived structure or poly fatty acid polyglycerides having two or more fatty acid-derived structures.

In the present invention, the (A) fatty acid polyglycerides essentially include two types having different viscosities, namely:

(a1) a fatty acid polyglyceride having a viscosity of 5000 mPa·s or higher at 25° C., and
(a2) a fatty acid polyglyceride having a viscosity lower than 5000 mPa·s at 25° C.

In this case, the viscosity of the (A) fatty acid polyglyceride is the maximum viscosity (mPa·s) measured at 25° C. using a Brookfield-type viscometer (spindle no. 7, rotation speed 5 rpm).

The high-viscosity (a1) fatty acid polyglyceride may be liquid, semi-solid or solid at ambient temperature (25° C.) as long as the viscosity is 5000 mPa·s or higher at 25° C. The viscosity is more preferably 10000 mPa·s or higher, and even more preferably 50000 mPa·s or higher. Additionally, one having a viscosity that is so high that the upper measurement limit is exceeded may even be used. However, if the polyglyceride is too hard, then it tends to become difficult to handle at the time of manufacture. Therefore, one having a hardness of 200 N or lower at 25° C. is preferable.

The hardness is a value (N) obtained by measuring the (a1) fatty acid polyglyceride at 25° C. with a rheometer (pressure-sensing shaft 5.6ϕ, needle insertion speed 2 cm/min, needle insertion level 3 mm) manufactured by Rheotech.

Furthermore, the high-viscosity (a1) fatty acid polyglyceride preferably has a molecular weight within the range 10 to 5000, and more preferably within the range 100 to 3000. Additionally, one in which the glycerin polymerization degree is within the range 2 to 11 is preferable, and one in which it is within the range 5 to 10 is more preferable.

Typical examples of the high-viscosity (a1) fatty acid polyglyceride include polyglyceryl-5 triisosterate, macadamia nut oil polyglyceryl-6 esters behenate, (isostearic acid/behenic acid) (glyceryl/polyglyceryl-6) esters, polyglyceryl-10 stearate, polyglyceryl-10 caprate and the like

As commercially available products, for example, Sunsoft A-193E-C (Taiyo Kagaku Co., Ltd.), S Face VL-211 (Sakamoto Yakuhin Kogyo Co., Ltd.), S Face VL-212 (Sakamoto Yakuhin Kogyo Co., Ltd.), Sunsoft Q-18Y-C (Taiyo Kagaku Co., Ltd.), Sunsoft Q-10Y-C (Taiyo Kagaku Co., Ltd.) and the like may be used. Component (a1) may be used as one type alone, or two or more types may be used.

On the other hand, the low-viscosity (a2) fatty acid polyglyceride has a viscosity lower than 5000 mPa·s at 25° C., preferably a viscosity of 3000 mPa·s or lower, and more preferably a viscosity of 1000 mPa·s or lower. The lower limit of the viscosity is not particularly limited.

Furthermore, the low-viscosity (a2) fatty acid polyglyceride preferably has a molecular weight within the range 10 to 5000, and more preferably within the range 500 to 4000. Additionally, one in which the glycerin polymerization degree is within the range 2 to 11 is preferable, and one in which it is within the range 2 to 10 is more preferable.

Typical examples of the low-viscosity (a2) fatty acid polyglyceride include polyglyceryl-2 triisosterate, polyglyceryl-2 diisostearate, polyglyceryl-10 laurate, polyglyceryl-10 nonaisostearate, polyglyceryl-2 dipolyhydroxystearate and the like.

As commercially available products, for example, Cosmol 43V (The Nisshin Oillio Group, Ltd.), WOGEL-18DV (Matsumoto Fine Chemical Co., Ltd.), Cosmol 42SV (The Nisshin Oillio Group, Ltd.), Sunsoft M-12J (Taiyo Kagaku Co., Ltd.), S Face IS-1000P (Sakamoto Yakuhin Kogyo Co., Ltd.), Dehymuls PGPH (BASF Corp.) and the like may be used. Component (a2) may be used as one type alone, or two or more types may be used.

The (a1) fatty acid polyglyceride content in the solid lip cosmetic of the present invention is 0.1% to 20% by mass, preferably 5% to 17.5% by mass, and more preferably 7.5% to 15% by mass. If the (a1) fatty acid polyglyceride content is less than 0.1% by mass, then the sense of resilience when applied and the sense of adhesion after being applied tend to become worse, and if more than 20% by mass is blended, then the cosmetic tends to become heavy to spread.

Additionally, the mass ratio ((a1):(a2)) between the (a1) fatty acid polyglyceride and the (a2) fatty acid polyglyceride is 1:0.5 to 1:5, preferably 1:1 to 1:2. If the mass ratio is outside said range, then the lightness of spreading, the lack of bleeding, the sense of adhesion and the sense of resilience tend to become inadequate.

### <(B) Silicic Anhydride>

The (B) silicic anhydride has an average particle size of 1 μm or smaller, preferably 5 to 100 nm (nanometers), and more preferably 10 to 20 nm. As long as the average particle size is within this range, one that is normally used in cosmetics may be used without any particular restrictions. If silicic anhydride having an average particle size greater than 1 μm is blended, then the sense of adhesion, the sense of resilience and the lack of bleeding cannot be adequately improved.

In the present specification, the “average particle size” is a value measured by a conventional method, such as the number-average size based on image analysis of transmission electron microscopy images.

Additionally, the (B) silicic anhydride may be subjected to a hydrophobic treatment. Although the hydrophobic treatment agent is not particularly limited, for example, organosilane-based compounds, silicone compounds, fluorine compounds and the like may be used. Specific examples of hydrophobically treated silicic anhydride include dimethylsilylated silica, trimethylsilylated silica, octylsilylated silica, silicone oil-treated silica, methylpolysiloxane-treated silica and the like.

As (B) silicic anhydrides that are commercially available products, for example, Aerosil #200, Aerosil R972, Aerosil R805, Aerosil RY200 (all from Nippon Aerosil Co., Ltd.), COSMO 55 (JGC Catalysts and Chemicals Ltd.) and the like may be favorably used.

The blended amount of the (B) silicic anhydride is preferably 0.25% to 4% by mass relative to the solid oil cosmetic, more preferably 0.5% to 3% by mass and even more preferably 1% to 2% by mass. If the blended amount of the (B) silicic anhydride is less than 0.25% by mass, then there are cases in which the bleeding cannot be prevented from occurring, and if more than 4% by mass is blended, then the stability over time is lost and the cosmetic becomes heavy to spread at the time of application and the luster tends to be suppressed.

### <(C) Colorant>

As the (C) colorant, one that is normally blended into lip cosmetics, such as a pigment or a pearlescent pigment, may be used.

Specifically, it is possible to select any one or more types from among inorganic white pigments (titanium dioxide and zinc oxide); inorganic red pigments (red iron oxide and iron titanate); inorganic brown pigments (γ-iron oxide); inorganic yellow pigments (yellow iron oxide and ocher); inorganic black pigments (black iron oxide, carbon and low-order titanium oxide); inorganic violet pigments (mango violet and cobalt violet); inorganic green pigments (chromium oxide, chromium hydroxide and cobalt titanate); inorganic blue pigments (ultramarine blue and Prussian blue); pearlescent pigments (titanium oxide-coated mica, titanium oxide-coated bismuth oxychloride, titanium oxide-coated talc, colored titanium oxide-coated mica, bismuth oxychloride and argentine); metal powder pigments (aluminum powder and copper powder); organic pigments (Red No. 202, Red No. 205, Red No. 220, Red No. 228, Red No. 405, Orange No. 203, Orange No. 204, Yellow No. 205, Yellow No. 401 and Blue No. 404); organic pigments that are zirconium, barium or aluminum lakes (Red No. 3, Red No. 104, Red No. 227, Red No. 401, Orange No. 205, Yellow No. 4, Yellow No. 202, Green No. 3, Blue No. 1); natural pigments (chlorophyll, carotenoids (β-carotene), carthamin, cochineal, chalcone, curcumin, betanin, flavonol, flavone, anthocyanidin, anthraquinone and naphthoquinone); and functional pigments (boron nitride, photochromic pigments, synthetic fluorophlogopite, iron-containing synthetic fluorophlogopite and fine-particle composite powders (hybrid fine powders)). However, the present invention is not limited to containing the above.

A powdered colorant that is not surface-treated may be used, or one subjected to a surface treatment by an organosilane-based compound, a silicone compound, a fluorine compound, a silane coupling agent, a fluororesin, a fatty acid, a fatty acid soap, lauroyl lysine or the like may be used.

The blended amount of the (C) colorant is not particularly limited, but should be 3% by mass or greater, and more preferably 5% to 20% by mass relative to the solid lip cosmetic. If the blended amount of the (C) colorant is less than 3% by mass, then sufficient coloring effects are difficult to obtain, and blending more than 20% by mass is unfavorable because the cosmetic tends to bleed and the like.

### <Optional Blended Components>

Aside from the above-mentioned components (A) to (C), oils other than those mentioned above, waxes, powders, polymer compounds, humectants, fragrances, antioxidants, preservatives, beauty care components, ultraviolet absorbing agents and the like, which are used in normal lip cosmetics, may be appropriately blended within a range not compromising the effects of the present invention.

In particular, oils other than those mentioned above are exemplified by ester oils such as diisostearyl malate, sucrose tetraisostearate, triisostearin, di(phytosteryl/octyldodecyl) lauroyl glutamate, diisopropyl sebacate, triethylhexanoin, isopropyl myristate, diethyl hexyl succinate and the like; hydrocarbon oils such as mineral oil, hydrogenated polyisobutene (hydrogenated isopolybutene), hydrogenated polydecene and the like; and silicone oils such as dimethicone and the like. However, the oils are not limited to the above.

Additionally, the waxes are not particularly limited as long as they are of a type blended into normal cosmetics. Examples include carnauba wax, candelilla wax, polyethylene wax, beeswax, ceresin, microcrystalline wax, solid paraffin, Japan wax and the like.

In the case in which a wax is blended, the blended amount thereof is preferably 0.1% to 20% by mass, more preferably 1% to 15% by mass, and even more preferably 3% to 12% by mass relative to the solid lip cosmetic. By blending 0.1% by mass or more of a wax, the stability over time tends to be further improved. However, if the blended amount of the wax exceeds 20% by mass, then there are cases in which the cosmetic becomes heavy to spread at the time of application.

The specific format of the solid lip cosmetic of the present invention need only be of a type that is directly applied to the lips, and may be of solid stick form or of solid paste form. A solid stick form is particularly preferred.

The solid lip cosmetic of the present invention can be manufactured in accordance with a method that has conventionally been used in solid cosmetics. In concise terms, it may be manufactured by heating and mixing all of the components, pouring the mixture into a mold or a container, then cooling to solidify.

The hardness of the solid lip cosmetic of the present invention is preferably 25 to 70 N, more preferably 35 to 65 N. If the hardness is too low, then problems occur in which the cosmetic crumbles at the time of application, and if the hardness is too high, then there are issues such as the cosmetic becoming heavy to spread and thus difficult to apply.

The hardness is a value (N) obtained by measuring a sample at 25° C. with a rheometer (pressure-sensing shaft 1ϕ), needle insertion speed 2 cm/min, needle insertion level 10 mm) manufactured by Rheotech.

### EXAMPLES

Although the present invention will be explained in further detail by providing examples below, the present invention is not limited in any way thereby. Where not otherwise noted, the blended amounts are indicated in percentage by mass relative to the system in which the relevant components are blended. Before specifically explaining each example, the evaluation methods that were employed will be explained.

### <Organoleptic Evaluation>

Samples of the examples and comparative examples were actually used by ten expert panelists and were evaluated regarding the lightness of spreading during application, the sense of resilience during application, the sense of adhesion after application, the luster after application and the lack of bleeding.

Each of the expert panelists performed a five-level organoleptic evaluation in accordance with the evaluation scoring criteria indicated below, and the samples were judged based on the evaluation criteria indicated below in accordance with the total score.

### Evaluation Scoring Criteria

5: Very good

### 4: Good

### 3: Normal

### 2: Poor

1: Very poor

### Evaluation Criteria

S: Total score of 45 or more points
A: Total score of 40 to 44 points
B: Total score of 30 to 39 points
C: Total score of 20 to 29 points
D: Total score of 10 to 19 points
E: Total score of 9 points or less

### <Stability Over Time>

The samples of the examples and comparative examples were measured for hardness after being placed at rest for one week in a cycle tester (−5 to 40° C., two cycles per day), then being left for 24 hours at 20° C. The samples were judged on the basis of the evaluation criteria indicated below based on the change in hardness from the initial hardness (the hardness after being left for 24 hours at 20° C. after being molded).

### Evaluation Criteria

S: Very slight hardness change
A: Slight hardness change
B: Some hardness change, but no problems such as crumbling when applied
C: Extreme hardness change, with problems such as crumbling when applied

### Examples 1 to 6 and Comparative Examples 1 to 10

Solid lip cosmetics having the compositions indicated in Table 1 and Table 2 below were prepared, and evaluated in the categories mentioned above.

TABLE 1

| | | | | | | | | Comp | Comp |
|---|---|---|---|---|---|---|---|---|---|
| | | Ex 1 | Ex 2 | Ex 3 | Ex 4 | Ex 5 | Ex 6 | Ex 1 | Ex 2 |
| Polyethylene wax | | 10 | 10 | 10 | 10 | 10 | 10 | 10 | 10 |
| Hydrogenated polyisobutene | | 15 | 15 | 15 | 15 | 15 | 15 | 15 | 15 |
| Hydrogenated polydecene | | 7 | 7 | — | 7 | 7 | 7 | 7 | 7 |
| Diisostearyl malate | | bal | bal | bal | bal | bal | bal | bal | bal |
| Di(phytosteryl/octyldodecyl) | | 7 | 7 | — | 7 | 7 | 7 | 7 | 7 |
| lauroyl glutamate | | | | | | | | | |
| (a2) | Polyglyceryl-2 | 10 | 10 | 50 | 10 | 10 | — | 10 | 10 |
| | triisostearate *1 | | | | | | | | |
| | Polyglyceryl-2 | — | — | — | — | — | 10 | — | — |
| | diisostearate *2 | | | | | | | | |
| Diphenylsiloxy phenyl trimethicone | | 20 | 20 | — | 20 | 20 | 20 | 20 | 20 |
| (a1) | Polyglyceryl-5 | 10 | — | 10 | 5 | 17.5 | 10 | — | — |
| | triisostearate *3 | | | | | | | | |
| | Macadamia nut oil | — | 10 | — | — | — | — | — | — |
| | polyglyceryl-6 esters | | | | | | | | |
| | behenate *4 | | | | | | | | |
| Dimethylsilylated silica (average | | 2 | 2 | 2 | 0.25 | 1 | 2 | — | — |
| particle size = 12 nm) | | | | | | | | | |
| Spherical silicic anhydride (average | | — | — | — | — | — | — | — | 2 |
| particle size = 10 μm) | | | | | | | | | |
| Colorant | | 7 | 7 | 7 | 7 | 7 | 7 | 7 | 7 |
| Total | | 100 | 100 | 100 | 100 | 100 | 100 | 100 | 100 |
| Hardness (N) | | 54 | 45 | 52 | 46 | 52 | 51 | 43 | 40 |
| (a1):(a2) | | 1:1 | 1:1 | 1:5 | 1:2 | 1:0.57 | 1:1 | — | — |
| Evaluation | Lightness of spreading | A | A | A | A | B | A | A | A |
| | Sense of resilience | S | A | S | B | S | S | E | E |
| | Sense of adhesion | S | S | S | A | S | S | D | D |
| | Luster level | A | S | A | A | A | A | A | A |
| | Lack of bleeding | A | A | A | B | A | A | D | D |
| | Stability | B | B | B | A | B | B | S | S |

TABLE 2

| | | Comp | Comp | Comp | Comp | Comp | Comp | Comp | Comp |
|---|---|---|---|---|---|---|---|---|---|
| | | Ex 3 | Ex 4 | Ex 5 | Ex 6 | Ex 7 | Ex 8 | Ex 9 | Ex 10 |
| Polyethylene wax | | 10 | 10 | 10 | 10 | 10 | 10 | 10 | 10 |
| Hydrogenated polyisobutene | | 15 | 15 | 15 | 15 | 15 | 15 | 15 | 15 |
| Hydrogenated polydecene | | 7 | 7 | 7 | 7 | 7 | 7 | 7 | 50 |
| Diisostearyl malate | | bal | bal | bal | — | bal | bal | bal | bal |
| Di(phytosteryl/octyldodecyl) | | 7 | 7 | 7 | bal | 7 | 7 | 7 | — |
| lauroyl glutamate | | | | | | | | | |
| (a2) | Polyglyceryl-2 | 10 | 10 | 10 | 10 | 10 | 10 | 10 | — |
| | triisostearate *1 | | | | | | | | |
| Diphenylsiloxy phenyl trimethicone | | 20 | 20 | 20 | 20 | 20 | 20 | 20 | — |
| (a1) | Polyglyceryl-5 | — | — | 10 | 22 | 10 | — | 10 | 10 |
| | triisostearate *3 | | | | | | | | |
| Dipentaerythrityl hexahydroxy | | — | — | — | — | — | 10 | — | — |
| stearate | | | | | | | | | |
| Dimethylsilylated silica (average | | — | 2 | — | 1 | 5 | 2 | — | 2 |
| particle size = 12 nm) | | | | | | | | | |
| Spherical silicic anhydride (average | | 4 | — | — | — | — | — | 2 | — |
| particle size = 10 μm) | | | | | | | | | |
| Colorant | | 7 | 7 | 7 | 7 | 7 | 7 | 7 | 7 |
| Total | | 100 | 100 | 100 | 100 | 100 | 100 | 100 | 100 |
| Hardness (N) | | 40 | 44 | 49 | 53 | 66 | 53 | 49 | 40 |
| (a1):(a2) | | — | — | 1:1 | 1:0.45 | 1:1 | — | 1:1 | — |
| Evaluation | Lightness of spreading | A | A | A | E | D | B | A | A |
| | Sense of resilience | E | D | D | A | A | D | D | S |
| | Sense of adhesion | D | C | C | A | A | C | D | S |
| | Luster level | B | A | A | A | D | B | A | A |
| | Lack of bleeding | C | A | D | A | S | A | D | A |
| | Stability | S | B | S | B | C | B | S | C |

*1 Cosmol 43V (The Nisshin Oillio Group Ltd.), viscosity at 25° C. = 350 mPa · s
*2 WOGEL-18DV (Matsumoto Fine Chemical Co., Ltd.), viscosity at 25° C. = 350 mPa · s
*3 Sunsoft A-193E-C (Taiyo Kagaku Co., Ltd.), viscosity at 25° C. = 147500 mPa · s
*4 S Face VL-211 (Sakamoto Yakuhin Kogyo Co., Ltd.), hardness at 25° C. = 160N

As indicated in the tables above, with the solid lip cosmetics containing a low-viscosity fatty acid polyglyceride and containing relatively large quantities of oil, good lightness of spreading and high luster were obtained at the same time. However, bleeding tended to occur in the finish after being applied, and the sense of adhesion and the sense of resilience were inadequate (Comparative Example 1).

Upon blending common spherical silicic anhydride having an average particle size of approximately 10 μm, which is used in normal cosmetics, in order to suppress bleeding, the bleeding was mostly unable to be improved (Comparative Examples 2 and 3). However, the occurrence of bleeding was clearly suppressed when fine silicic anhydride having an average particle size of 1 μm or smaller was blended. Nevertheless, the sense of adhesion and the sense of resilience were still not adequate (Comparative Example 4).

Therefore, upon blending a high-viscosity fatty acid polyglyceride having a viscosity at 25° C. of 5000 mPa·s or more aside from the fine silicic anhydride, high evaluation results were exhibited in all categories (Examples 1 to 6). Considering that the lack of bleeding, the sense of adhesion and the sense of resilience were not improved by blending only a high-viscosity fatty acid polyglyceride without blending fine silicic anhydride (Comparative Example 5), these improvement effects were entirely unexpected and surprising.

On the other hand, it was observed that, if the blended amount of the high-viscosity fatty acid polyglyceride is too high, then the cosmetic becomes heavy to spread (Comparative Example 6). Additionally, when the blended amount of the fine silicic anhydride was too high, the cosmetic became heavy to spread, the luster was lost, the structure became brittle and the hardness decreased, and the stability over time became much worse (Comparative Example 7).

Additionally, one of the sense of adhesion, the sense of resilience and the lack of bleeding was inadequate in the case in which the high-viscosity fatty acid polyglyceride was replaced by blending dipentaerythrityl hexahydroxy stearate, which is a polar oil having a similar viscosity, and in the case in which the fine silicic anhydride was replaced by blending a common spherical silicic anhydride having an average particle size of approximately 10 μm (Comparative Examples 8 and 9). Furthermore, in the case in which a low-viscosity fatty acid polyglyceride was not included, the stability over time was inadequate (Comparative Example 10).

1. A solid lip cosmetic containing:
(A) fatty acid polyglycerides;
(B) silicic anhydride having an average particle size of 1 μm or smaller; and
(C) a colorant;
wherein
the component (A) includes
(a1) a fatty acid polyglyceride having a viscosity of 5000 mPa·s or higher at 25° C., and
(a2) a fatty acid polyglyceride having a viscosity lower than 5000 mPa·s at 25° C.;
the component (a1) content is 0.1% to 20% by mass;
the mass ratio between components (a1) and (a2) satisfies (a1):(a2)=1:0.5 to 1:5; and
the component (B) content is 0.25% to 4% by mass.

2. The solid lip cosmetic according to claim 1, containing 0.1% to 20% by mass of a wax.

3. The solid lip cosmetic according to claim 1, wherein the
(a1) fatty acid polyglyceride is of one or more types selected from the group consisting of polyglyceryl-5 triisosterate, macadamia nut oil polyglyceryl-6 esters behenate, (isostearic acid/behenic acid) (glyceryl/polyglyceryl-6) esters, polyglyceryl-10 stearate and polyglyceryl-10 caprate.

4. The solid lip cosmetic according to claim 1, wherein the (a2) fatty acid polyglyceride is of one or more types selected from the group consisting of polyglyceryl-2 triisosterate, polyglyceryl-2 diisostearate, polyglyceryl-10 laurate, polyglyceryl-10 nonaisostearate and polyglyceryl-2 dipolyhydroxystearate.

5. The solid lip cosmetic according to claim 1, which is in stick form.
